(19) United States
(12) Patent Application Publication
(10) Pub. No.: US 2007/0014883 A1
(43) Pub. Date: Jan. 18, 2007

(54) Method for treating osteoarthritis

(76) Inventor: Peter Rohdewald, Munster (DE)

Correspondence Address: Eric Sinn, Esq.;Mintz, Levin, Cohn, Ferris, Glovsky and Popeo, P.C, The Chrysler Center, 666 Third Avenue, 24th Floor, New York, NY, 10017, US

(21) Appl. No.: 11/181,198

(22) Filed: Jul. 13, 2005

(51) Int. Cl. A61K 31/7048; A61K 31/353; A61K 36/13

(52) U.S. Cl. 424/770; 514/27; 514/456

(57) ABSTRACT

The invention relates to methods for treating osteoarthritis by administering a composition consisting essentially of proanthocyanidins to a patient suffering from such a disorder.

### FIELD OF THE INVENTION

The invention concerns proanthocyanidins pharmaceutical formulations and their use for treating osteoarthritis.

### BACKGROUND

Osteoarthritis (OA) of the knee is the most common joint disorder inducing pain and stiffness. Heightened inflammatory mediators (cytokines, prostaglandins, nitric oxide, and proteases) perpetuate cartilage damage that ensues from repeated mechanical injury. Reduction of symptoms is an important goal that is only partially achieved with current therapies that recently have been recognized to have serious side effects.

More than 15 million Americans suffer from rheumatoid and osteoarthritis (OA). Much of adult disability is due to OA of the knee, the most common joint disorder. The Chingford study documented a 12% prevalence of radiological knee OA and 6% prevalence of symptomatic knee OA in women aged 45-64 years (1). The age-and-sex standardized incidence rate for knee OA is approximately 240 of 100,000 person/year (95%, CI: 218-162) (2). Although OA is not considered an inflammatory disease, mediators classically associated with inflammation (cytokines, prostaglandins, nitric oxide, proteases) perpetuate cartilage damage that ensues from repeated mechanical injury. Cartilage destruction is an important pathological feature and a major cause of joint dysfunction. It is caused in two ways: intrinsic, where chondrocytes are responsible for the degradation of the extra-cellular matrix; and, extrinsic, where cells and tissues other than chondrocytes, such as inflamed synovium, pannus tissue, and inflammatory cells affect the extra cellular matrix via synovial fluids (3). Matrix metalloproteinases (MMP) are expressed in osteoarthritic cartilage, producing tissue damage (5, 6) and the resulting pain. Nitric oxide (NO) is a major catabolic factor produced by chondrocytes in response to proinflammatory cytokines such IL-1β and tumor necrosis factor-α (10). Non-steroidal anti-inflammatory drugs (NSAIDS) are the most commonly used medications for arthritis. However, recent studies have shown significant side effects including gastrointestinal problems (26).

Proanthocyanidins, especially proanthocyanidins in the form of Pycnogenol®, have been described in numerous references including, for example, Passwater, R. A. The New Superantioxidant Plus, Keats Publishing Inc., New Canaan, Conn. USA, 1992, Passwater, R. A., All About Pycnogenol, Avery Publishing Group, Garden City Park, N.Y., 1998, Passwater, R. A. and Kandaswami, C., Pycnogenol The Super Protector Nutrient, Keats Publishing Inc., New Canaan, Conn. USA, 1994, Passwater, R. A. Pycnogenol for Superior Health, McCleery and Sons Publishing, Fargo, N.D. USA, 2001, and Passwater, R. A. Pycnogenolfor Superior Health, Editions Stylum, Switzerland, 2001.

U.S. Pat. No. 6,346,547 refers to the use of an amino acid based compound and proanthocyanidins for treating a range of illnesses including atherosclerosis, coronary artery disease, restenosis, osteoarthritis, reperfusion injury from blood clots, organic repair or organ transplants, neurodegenerative disease and stroke. No data was presented that indicates an effectiveness of the treatment for osteoarthritis. Significantly, no data was presented to show that proanthocyanidins by itself, in the absence of any other active ingredient, has an effect on any disorder.

U.S. Pat. No. 6,656,925 refers to the use of compositions compromising an inhibitor of nitric oxide production, an aminosugar and optionally proanthocyanidins for treating arthritis. There is no data, suggestion, or teaching that proanthocyanidins by itself has an effect on arthritis.

U.S. Pat. No. 6,469,053 refers to the use of methylated cocoa procyanidins for treatment of chronic arthritis. However, the '053 patent provides no experimental data showing that proanthocyanidins have an effect on arthritis and osteoarthritis sufferers. Further, there is no evidence that unmethylated proanthocyanidins have an effect on osteoarthritis.

Other published patent application that refers to proanthocyanidins as part of a composition for treating arthritis include US20040162269, US20030185907, US20020119952 and WO2003084532. Significantly, none of these applications teach or suggest that proanthocyanidins by itself (i.e., without another active ingredient) is effective for the treatment of osteoarthritis. Further, our review of the scientific literature did not find any reference that provides data on the efficacy of proanthocyanidins alone for the treatment of osteoarthritis.

### BRIEF DESCRIPTION OF THE INVENTION

The present invention relates to methods and compositions for preventing, treating or providing relief from the symptoms of osteoarthritis, and other degenerative joint diseases. These symptoms include pain, stiffness and lack of function from the affected joint.

One embodiment of the invention is directed to a method for treating osteoarthritis in a patient. The method involve the step of administering to the patient a therapeutically effective amount of a composition consisting essentially of proanthocyanidins. That is, the composition contains a single active ingredient—proanthocyanidins. The method may reduce at least one symptom of osteoarthritis. These symptoms include, at least, such as joint pain, joint stiffness, or joint inflammation in said patient.

The amount of proanthocyanidins administered may be, for example, between 20 mg to 10 grams a day, between 50 mg to 3 grams a day, 50 to 500 mg a day, or between 100 mg to 500 mg per day. Administration may be continuous (e.g., daily or 2, 3 or 4 times a day) for a period of at least 30 days, at least 60 days, or at least 90 days.

In another aspect, the proanthocyanidin used in the method of the invention is Pycnogenol®. In a preferred embodiment, the proanthocyanidins is at least about 30% or at least 50% by weight of said composition.

The proanthocyanidins may be administered in the form of a pill, tablet, caplet or capsule. In addition, the proanthocyanidins may be a food. The food may be a liquid (such as a tea or a beverage), a food additive or a spice.

In a preferred embodiment, the method of the invention reduces the need for analgesic in a patent which suffers from osteoarthritis. In one aspect, the analgesic is a NSAID inhibitor or COX-2 inhibitor.

### DETAILED DESCRIPTION OF THE INVENTION

The invention provides a method for treating various symptoms associated with osteoarthritis. The term “treating” in its various grammatical forms in relation to the present invention refers to preventing, curing, reversing, attenuating, alleviating, ameliorating minimizing, suppressing, or halting the deleterious effects of osteoarthritis, osteoarthritis progression or one of the symptoms of osteoarthritis. Symptoms of osteoarthritis include, but are not limited to, pain (including joint pain), stiffness, limited joint movement, swelling, and bony enlargement. These symptoms may manifest themselves in various parts of a patient such as, for example, hip, knee, spine, hands, or any other joint in the body. Furthermore, these symptoms may manifest during certain activities such as bending, kneeling, stair climbing, running, rowing, and other strong or extended physical exertion, pain and stiffness in a joint during or after use, or after a period of inactivity, or any combination of these. In addition, the symptom may be related to weather—such as discomfort in a joint before or during a change in the weather (a drop in barometric pressure).

In this study, we investigated the activity of proanthocyanidins. Proanthocyanidins designates a group of flavanoids that includes the subgroups procyanidins, prodelphinidins and propelargonidins. Proanthocyanidins are homogeneous or heterogeneous polymers consisting of the monomer units catechin or epicatechin, which are connected either by 4-8 or 4-6 linkages, to the effect that a great number of isomer proanthocyanidins exist. Typically, the proanthocyanidins oligomers have a chain length of 2-12 monomer units. Proanthocyanidins may be synthesized or extracted from a plant material. Nonlimiting examples of plant material sources of proanthocyanidins include grape seeds, grape skin, pine barks, ginkgo leaves, peanuts, and cocoa beans, tamarind, tomato, peanut, almond, apple, cranberry, blueberry, tea leaves.

Every plant species has its own unique proanthocyanidins mix. In a preferred embodiment, the proanthocyanidins are derived from pine bark. A well-known product containing proanthocyanidins, which is available in trade as a preparation of a food supplement under the name Pycnogenol®, is an extract of the maritime pine bark (Pinus pinaster). Pycnogenol®, the extract from French maritime pine bark (Pinus pinaster) is a registered trademark belonging to Horphag Research, Ltd. The Pycnogenol® food supplement contains approximately 70-80% of proanthocyanidins and is a complex mixture of phenolic substances. Besides proanthocyanidins and its monomeric unit catechin, Pycnogenol® food supplement contains taxifolin and a wide range of phenolic acids, e.g. free acids like p-hydroxybenzoic acid, protoacatechic acid, vanillic acid, caffeic acid and ferulic acid as well as its glucosides and glucose esters (20). Most of the positive effects of Pycnogenol® are attributed to its antioxidant qualities.

Pycnogenol® has anti-inflammatory effects (21) and antioxidant activity (22). It has strong free radical-scavenging activity against reactive oxygen and nitrogen species (22) and modulates the production of NO radicals in activated macrophages by quenching the NO radical and inhibiting both iNOS and mRNA expressions and iNOS activity (23). The two major metabolites developing from ingested Pycnogenol® in humans (δ-(3,4-dihydroxy-phenyl)-γ-valerolactone (M1) and δ-(3-methoxy-4-hydroxyphenyl)-γ-valerolactone (M2)) display strong inhibitory activity toward matrix-metallo-proteases MMP-1, MMP-2, and MMP-9 (24). In consideration of the strong antioxidant and anti-inflammatory profile of Pycnogenol® and its strong inhibitory activity toward MMPs, the studies in the Examples were conducted to assay a possible efficacy of Pycnogenol® in osteoarthritis of the knee.

It was found, surprisingly, that the oral administration of a source of proanthocyanidins reduces the symptoms of osteoarthritis. This result has not been reported in the scientific literature. Perhaps this effect has not been reported because the maximal effect of proanthocyanidins is not apparent until after 60 or even 90 days and studies of this length, using proanthocyanidins alone, has not been performed. In one preferred embodiment, the proanthocyanidins are administered daily for a period over 30, 60, 90, or 120 days. This administration may be continued indefinitely. Over time, the cumulative effect of proanthocyanidins administration leaves a patient with fewer and less severe symptoms of osteoarthritis. As a direct consequence, the methods of the invention reduces the need for analgesics such as NSAIDs or COX-2 inhibitors. These analgesics have been associated with one or more side effects such as increasing joint deterioration, inhibiting cartilage formation, and detrimental cardiovascular effects.

In the most preferred embodiment, the proanthocyanidins are administered in a composition representing the only active ingredient of the composition. For example, the proanthocyanidins are administered in the absence of other medicaments such as glucosamine, chondroitin, or vitamins. In a preferred embodiment, the proanthocyanidins (such as delivered by Pycnogenol®) are the only antioxidant in the composition.

### EXAMPLE

### Example 1

### Treatment of Osteoarthritis with Procyanidins

### Study Design

Patients and Methods

A total of 40 knee osteoarthritic patients (37 female and 3 male) were enrolled in this prospective, randomized, parallel group double blind study. Of these 5 were dismissed due to non-compliance and 35 were enrolled in the study. Forty patients were enrolled in the study. Twenty patients (2 male and 18 female) aged between 36 and 61 years (mean±SD: 47.5±7.4 years), received Pycnogenol®, while the other 20 patients (1 male and 19 female) aged between 29 and 63 years (48.9±9.6 years), received the placebo. All patients fulfilled the American College of Rheumatology radiological and clinical criteria for knee OA (25). Inclusion criteria were as follows: Age between 25-65 years; primary OA of the knee (grade 1 or 2); pain in the target knee for minimum of 3 months; the use of NSAIDs or COX-2 inhibitors; and, informed consent form signed by the subject. Exclusion criteria: secondary OA (owing to previous trauma); arthroscopy of the target knee performed less than 6 months prior to enrollment or during the trial period; or, other chronic inflammatory process.

Patients received a monthly dose of Pycnogenol® (three 3 tablets with 50 mg Pycnogenol® each) or 3 placebo tablets. Patients also received 4 copies of the Western Ontario McMaster Universities Osteoarthritis Index (WOMAC) form, containing twenty-four 10 mm visual analogue scales to assess pain, stiffness, and physical function, and a composite. High scores indicate greater disease severity. Each subject was contacted by telephone once every 7 days to verbally complete the WOMAC form. Each patient visited the trial center every 30 days (maximum, 33 days), with the final visit to be accomplished after 12 weeks of using the Pycnogenol® or placebo. Medications were counted and the WOMAC forms evaluated exactly every 30 days. Each patient's intake of NSAIDs, COX-2 inhibitors, or other drugs, was catalogued in each patient's diary, and drug use was counted at each visit to the trial center. Routine blood examinations were conducted on day 0 and day 90, to include red blood cell count, hemoglobin, white blood cell count and platelets. Biochemical parameters included alanine aminotransferase, AST, UREA, creatinin and fasting plasma glucose done by the clinical laboratory of Mashhad Medical School.

Data analysis was performed with SPSS, version 11.5. The results were expressed as a mean±standard deviation. Parametric data were compared using students' t-test. Differences between groups were determined using X square tests. A P-value below 0.050 was considered significant.

Results

No side effects were reported during the study by patients in either group. There were no statistically significant differences between the groups in hematology and blood chemistry at the beginning or end of the study.

The WOMAC OA Index composite score measured no statistically significant difference between the placebo and Pycnogenol® supplemented groups prior to and after 1 month of treatment based upon self-reported pain, stiffness, and physical function (Table 1). Significant reductions in self-reported pain, physical function and WOMAC composite scores occurred in the Pycnogenol® treated groups after 2 months of supplementation compared to patients treated with placebo (Table 1).

TABLE 1
Pain, stiffness and physical function scores

| | Pycnogenol | Placebo | |
|---|---|---|---|
| Treatment | Mean ± SD | Mean ± SD | p value |
| | PAIN SCORE | | |
| 0 days | 292.30 ± 101.16 | 301.00 ± 119.62 | <0.805 |
| 30 days | 225.50 ± 116.45 | 251.41 ± 132.86 | <0.543 |
| 60 days | 165.00 ± 82.51 | 264.82 ± 129.18 | <0.010 |
| 90 days | 164.66 ± 72.46 | 306.00 ± 103.53 | <0.001 |
| | STIFFNESS SCORE | | |
| 0 days | 110.30 ± 66.10 | 120.15 ± 63.44 | <0.633 |
| 30 days | 86.22 ± 56.83 | 82.64 ± 62.66 | <0.861 |
| 60 days | 75.38 ± 57.98 | 92.52 ± 61.34 | <0.402 |
| 90 days | 75.50 ± 54.58 | 108.82 ± 56.85 | <0.086 |
| | PHYSICAL FUNCTION SCORE | | |
| 0 days | 997.90 ± 352.58 | 1042.00 ± 420.08 | <0.721 |
| 30 days | 707.33 ± 331.99 | 912.94 ± 481.88 | <0.149 |
| 60 days | 512.00 ± 272.87 | 909.52 ± 458.23 | <0.004 |
| 90 days | 485.50 ± 346.22 | 1014.00 ± 385.16 | <0.001 |

TABLE 1

| | WOMAC COMPOSITE SCORE | | |
|---|---|---|---|
| | Pycnogenol | Placebo | |
| Treatment | Mean ± SD | Mean ± SD | p value |
| 0 days | 1400.50 482.06 | 1463.15 552.31 | <0.704 |
| 30 days | 1019.05 463.01 | 1247.00 461.32 | <0.235 |
| 60 days | 752.38 347.44 | 1266.88 620.60 | <0.004 |
| 90 days | 725.50 346.22 | 1455.82 509.13 | <0.001 |

At the end of month 2 significant differences between the Pycnogenol® and placebo treated groups were observed for self-reported pain (p<0.010), physical function (p<0.004), and composite WOMAC (p<0.004) scores (Table 1).

After 3 months of supplementation with Pycnogenol® that group showed reductions of 43, 35, 52, and 49%, respectively, as to self-reported pain, stiffness, physical dysfunction, and composite WOMAC scores. The placebo group demonstrated only reductions of 4, 15, 5, and 6%, respectively, as to self-reported pain, stiffness, physical dysfunction and composite WOMAC scores by month 3 of supplementation (Table 2).

TABLE 2
Change in WOMAC scores after 60 and 90 days of Pycnogenol ® Therapy

| | | Amount of | | Amount of | | |
|---|---|---|---|---|---|---|
| | | change after | | change after 90 | | Change (%) |
| WOMAC | | 60 days | P | days | P | After 90 |
| Score | Treatment | Mean ± SD | value | Mean ± SD | value | days |
| Pain | Pycnogenol ® | 125.0 ± 93.2 | <.001 | 125.3 ± 81.5 | <.001 | 43 |
| | Placebo | 54.8 ± 109.7 | <.056 | 13.6 ± 71.1 | <.442 | 4 |
| Stiffness | Pycnogenol ® | 40.1 ± 60.6 | <.020 | 40.0 ± 59.1 | <.011 | 35 |
| | Placebo | 35.3 ± 44.4 | <.005 | 19.0 ± 46.4 | <.111 | 15 |
| Physical | Pycnogenol ® | 498.8 ± 334.6 | <.001 | 525.4 ± 316.3 | <.001 | 52 |
| Function | Placebo | 191.8 ± 281.6 | <.013 | 60.3 ± 200.6 | <.233 | 5 |
| Composite | Pycnogenol ® | 663.9 ± 435.0 | <.001 | 690.8 ± 408.1 | <.001 | 49 |
| | Placebo | 281.8 ± 403.4 | <.011 | 92.9 ± 301.7 | <.222 | 6 |

After 1 month of supplementation the number of NSAIDS and COX-2 inhibitor drugs used by the patients in both groups was unchanged. However after 2 and 3 months of Pycnogenol® consumption drug use was significantly reduced (p<0.001) (Table 3). On the other hand the placebo group significantly increased its use of NSAIDS and COX-2 inhibitors (p<0.037) by month 3 of intake. While no change was observed after 1 month of supplementation in the number of days that patients used NSAIDS and COX-2 inhibitors in either group by the completion of 2 months number of NSAIDS pills was significantly reduced (p<0.001) in the Pycnogenol® group (Table 4). The number of days the placebo group used NSAIDS and COX-2 inhibitors increased significantly (p<0.001).

TABLE 3
Change in the number of pills of NSAIDS or COX-2 inhibitory
drugs used per patient/month

| Drug Intake | | | | |
|---|---|---|---|---|
| Difference | Pycnogenol ® | | Placebo | |
| by Month | Mean ± SD | P value | Mean ± SD | P value |
| Months 1-2 | −8.3 ± 8.3 | <.001 | +0.9 ± 6.4 | <.551 |
| Months 2-3 | −6.3 ± 11.3 | <.030 | +2.6 ± 3.3 | <.005 |
| Months 1-3 | −14.7 ± 13.2 | <.001 | +3.6 ± 6.5 | <.037 |

TABLE 4
Change in the number of days using NSAIDS or COX-2 inhibitory
drugs/month

| Drug Intake | | | | |
|---|---|---|---|---|
| Difference | Pycnogenol ® | | Placebo | |
| by Months | Mean ± SD | P value | Mean ± SD | P value |
| Months 1-2 | −3.6 ± 4.7 | <.005 | +3.3 ± 8.5 | <.007 |
| Months 2-3 | −2.7 ± 5.3 | <.005 | +2.2 ± 4.1 | <.005 |
| Months 1-3 | −6.3 ± 5.8 | <.001 | +5.5 ± 6.8 | <.001 |

This study clearly showed that a concentrate of proanthocyanidins lowered clinical symptoms of osteoarthritis. In particular the regimen of NSAIDS and COX-2 inhibitors was decreased, demonstrating the clinical benefits from the dietary supplement. Self-reported pain, stiffness, physical function and overall WOMAC scores all gradually improved and reached statistical significance from 60 days onwards of supplementation with proanthocyanidins compared to placebo.

This result is surprising in that proanthocyanidins, administered as the only active ingredient, are able to induce a beneficial effect on patients suffering from osteoarthritis. As far was we can tell, no experimental data showing a beneficial effect of administering Pycnogenol alone on OA has ever been reported.

In vitro, proanthocyanidins had significant activity in lowering NO production by activated macrophages (23), resulting from inhibition of NF-κB controlled inducible NO synthetase (iNOS) expression and demonstrated to lower generation of peroxides from activated macrophages (“oxidative burst”) (Erben Bayeta et al., 2000). A significantly lowered generation of reactive oxygen species by peripheral neutrophils was found in Lupus erythematosus patients in response to Pycnogenol administration (29).

A lowering of inducible nitric oxide by proanthocyanidins (20-24) helps to explain symptom improvement in OA. NO is a major catabolic factor produced by chondrocytes in response to proinflammatory cytokines (10). Over-production of NO by chondrocytes plays a major role in the perpetuation of cartilage destruction in OA (11,12). Increased concentrations of nitrites have been demonstrated in synovial fluids of patients with OA, and iNOS has been demonstrated in OA synoviocytes and chondrocytes (13,14). NO and reactive oxygen species, combined to nitro peroxides, exert multiple effects on chondrocytes that promote the degradation of articular cartilage including activation of MMPs and apoptosis (15,16). Chondrocyte apoptosis is a particular feature of OA and studies implicated that NO and oxidative stress are important mediators in this process (17). Importantly NO levels are unchanged in patients without degenerative alterations. NO levels in fluid taken from painful joints are higher than in joints without pain. Thus reduction of NO and reactive oxygen species by Pycnogenol® should reduce tissue damage and this helps to explain the lowered clinical symptoms of OA.

Osteoarthritic chondrocytes release matrix metalloproteinases (MMP) which promote cleavage of articular collagen and gelatin. Excessive MMP expression exacerbates articular connective tissue and cartilage degradation and plays a critical role in the development of inflammatory joint disease. A broad spectrum of MMP species are expressed in osteoarthritic cartilage and these contribute significantly to tissue damage, pain and stiffness (4-9).

Human metabolites of proanthocyanidins, δ-(3,4-dihydroxy-phenyl)-γ-valerolactone (M1) and δ-(3-methoxy-4-hydroxyphenyl)-γ-valerolactone (M2), were shown to be more potent than the parent molecules for inhibition of MMP-1, MMP-2 and MMP-9 (Grimm et al. 2004) (24). The inhibitory activity of proanthocyanidins metabolites M1 and M2 against MMP-2 and MMP-9 was found to be 100-fold more potent than that of captopril, a well documented inhibitor of these MMPs. Moreover, proanthocyanidins metabolites were as potent as hydrocortisone for inhibition of MMP-9 release from activated macrophages.

The pronounced MMP inhibitory activity of proanthocyanidin and its metabolites should provide significant help for allowing recovery of articular tissue. From our experiments it was striking that all clinical symptoms improved gradually during the three months treatment course. It may be speculated that anti-inflammatory and MMP-inhibitory activity of proanthocyanidins allowed for progressive articular tissue recovery which resulted in significant symptom improvement only after 2 months treatment.

In conclusion, the anti-inflammatory and tissue-protective activity of proanthocyanidins reduced symptoms of OA in a double blind placebo controlled trial. Therefore, dietary supplementation with proanthocyanidins offers promise an alternative method for relieving osteoarthritis symptoms pain and stiffness.

### REFERENCES

1 Hart D J, Doyle D B, Spector T D. 1995. Association between metabolic factors and knee osteoarthritis in women: the Chingford study. J Rheumatol, 22:1118-23.

2 Oliveria S A, Felson D T, Reed J I, et al. 1995. Incidence of symptomatic hand, hip and knee osteoarthritis among patients in a health maintenance organization. Arthritis Rheum, 38:1134-41.

3 Yashihara Y, Nakamura H, Obata K, et al. 2000. Matrix metalloproteinases and tissue inhibitors of metalloproteinases in synovial fluids from patients with rheumatoid arthritis or osteoarthritis. Ann Rheum Dis, 59:455-61.

4 Sholpov B V, Lie W R, Mainardi C L, et al. 1997. Osteoarthritic lesions: involvement of three different collagenases. Arthritis Rheum, 40:2065-74.

5 Imai K, Ohata S, Matsumoto T, et al. 1997. Expression of membrane-type human osteoarthritic cartilage. Am J Pathol, 151:245-56.

6 Mohtai M. Smith R L, Schurman D J, et al. 1992. Expression of 92-KD type 4 collagenase/gelatinase (gelatinase B) in osteoarthritic cartilage and sinovium. Lab Invest, 66:680-90.

7 Okada Y, Shinmei M, Tanaka O, et al. 1992. Localization of matrix metallproteinase 3 (stromelysin) in human osteoarthritic cartilage. Lab Invest, 66:680-90.

8 Ohta S, Imai K, Yamashita K, et al. 1998. Expression of matrix metallproteinase 7 (matrilysin) in human osteoarthritic cartilage. Lab Invest, 78:79-87.

9 Mitchell P G, Magna H A, Reeves L M, et al. 1996. Cloning, expression and type II collagenolytic activity of matrix metallopreoteinase-13 from osteoarthritic cartilage. J Clin Invest, 97:761-68.

10 Pelletier J P, Martel-Pelletier J, Abramson S B. 2001. Osteoarthritis, an inflammatory disease: potential implication for the selection of new therapeutic targets. Arthritis Rheum, 44:1237-47.

11 Pelletier J P, Mineau F, Ranger P, et al. 1996. The increase synthesis of inducible nitric oxide inhibits IL-1ra synthesis by articular chondrocytes; possible role in osteoarthritic cartilage degradation. Osteoarthritis Cartilage, 4:77-84.

12 Grabowski P S, Wright P K, Vanat Hof R J, et al. 1997. Immunolocalization of inducible nitric oxide synthase in synovium and cartilage in rheumatoid arthritis and osteoarthritis. Br J Rheumatol, 36:651-55.

13 Hayashi T, Abe E, Yamate T, et al. 1997. Nitric oxide production by superfacial and deep articular chondrocytes. Arthritis Rheum, 40:261-69.

14 Loeser R F, Carlson C S, Del Carlo M, et al. 2002. Detection of nitrotyrosine in aging and osteoarthritic cartilage: correlation of oxidative damage with the presence of interleukin-1β and with chondrocyte resistance to insulin-like growth factor 1. Arthritis Rheum, 46:2349-57.

15 Abramson S B, Attur M. Amin A R, et al. 2001. Nitric oxide and inflammatory mediators in the perpetuation of osteoarthritis. Curr Rheumatol Rep, 3:535-41.

16 Hirai Y, Migita K, Honda S, et al. 2001. Effects of nitric oxide on matrix metallprotenaise-2 production by rheumatoid synovial cells. Life Sci, 2001;68:913-20.

17 Lotz, M. 1999. The role of nitric oxide in articular cartilage damage. Rheum Dis Clin Norh Am, 25:269-82.

18 DiCesare P E, Abramson S B. 2005. Pathogenesis of osteoarthritis. In: Kelley's textbook of Rheumatology, Harris E D, ed. Elsevier Saunders, Philadelphia: 1493-1513.

19 Bates-Smith, E C. 1975. Phytochemistry of proanthocyanidins. Phytocehmistry, 14:1107-13.

20 Rohdewald P. 2002. A review of the French maritime pine bark extract (Pycnogenol®), a herbal medication with a diverse clinical phamacology. Int J Clin Pharmacol Therap, 40 (4):158-168.21

21 Blazso G, Gabor M, Sibbel R, et al. 1994. Anti-inflammatory and super-oxide radical scavenging activities of procyanidin-containing extracts from bark of Pinus pinastersol and its fractions. Pharm Pharmacol, 3:217-20.

22 Packer L, Rimbach G, Virgili F. 1999. Antioxidant activity in activated macrophages by procyanidins extracted from pine (Pinus maritime) bark (Pycnogenol®). Free Radic Biol Med, 704-24.

23 Virgili F, Kobuchi H, Packer L. 1998. Nitrogen monoxide metabolism: antioxidant properties and modulation of inducible NO synthase activity in activated macrophages by procyanidins extracted from pinus maritime (Pycnogenol®). In: Flavonoids in health and disease. Rice-Evans C A, Packer L M, eds. Marcel Dekker: New York. 421-36.

24 Grimm T, Schafer A, Högger P. Antioxidant activity and inhibition of matrix metalloproteinases by metabolites of maritime pine bark extract (Pycnogenol®). 2004. Free Radic Biol, 36:811-22.

25 Altman R, Asch E, Bloch D, et al. 1986. Development of criteria for the classification of osteoarthritis of the knee. Arthritis Rheum, 29:1039-49.

26. Spiegel B M R, Chiou C F, Ofman J J. 2005. Minimizing complications from nonsteroidal anti-inflammatory drugs: Cost-effectiveness of competing strategies in varying risk groups. Arthritis Rheumat, 53:185-197

27. Saliou C, Rimbach G, Moini H, et al. 2001. Solar ultraviolet-induced erythema in human skin and nuclear factor-kappa-B dependent gene expression in keratinocytes are modulated by a French maritime pine bark extract. Free Radic Biol Med, 30:154-160

28. Bayeta E, Benjamin M S and Lau B H S. Pycnogenol inhibits generation of inflammatory mediators in macrophages. Nutrition Research 20: 249-259, 2000.

29. Stefanescu M, Matache C, Onu A, Tanaseanu S, Dragomir C, Constantinescu I, Schönlau F, Rohdewald P, Szegli G. Pycnogenol® efficacy in the treatment of systemic Lupus erythematosus patients. Phytother Res 15: 698-704, 2001.

All patents, patent applications and references in this Specification are incorporated by reference herein.

We claim:

1. A method for treating osteoarthritis in a patient comprising the step of administering to said patient a therapeutically effective amount of a composition consisting essentially of proanthocyanidins.

2. The method of claim 1 wherein said method reduces joint pain, joint stiffness, or joint inflammation in said patient.

3. The method of claim 1 wherein about 20 mg to about 10 grams per patient per day of proanthocyanidins are administered.

4. The method of claim 1 wherein about 50 mg to 500 mg per patient per day of proanthocyanidins are administered.

5. The method according to claim 3 comprising administration of said composition taken on a periodic basis each day over the course of a treatment period.

6. The method of claim 1 wherein said administrating comprises administering said proanthocyanidins daily for a period of at least 30 days, at least 60 days, at least 90 days or at least 120 days.

7. The method of claim 1 wherein the proanthocyanidins are an extract from a plant material.

8. The method of claim 7 wherein the proanthocyanidins are from pine bark extract.

9. The method of claim 1 wherein proanthocyanidins are obtained by synthesis.

10. The method of claim 1 wherein said composition is administered as a tablet, capsule, food additive or beverage.

11. The method of claim 1 wherein said method reduces the frequency and severity of osteoarthritis symptoms.

12. The method of claim 1 wherein said method reduces the frequency and amount of analgesics administered to said patient.

13. The method of claim 1 wherein said proanthocyanidins are present in an aggregate amount of at least about 20-100% by weight of said composition.

14. The method according to claim 1 wherein said proanthocyanidins are present in an aggregate amount of at least about 50% by weight of said composition.

15. The method of claim 1 wherein said proanthocyanidins is the only active ingredient administered to said patient.
